(19) United States
(12) Patent Application Publication
(10) Pub. No.: US 2007/0298147 A1
(43) Pub. Date: Dec. 27, 2007

(54) METHOD AND APPARATUS FOR TRACKING PROCESSED MEAT PROVIDED TO THE PUBLIC FROM THE TIME THE RAW MEAT WAS HANDLED BY AN INITIAL PRODUCTION FACILITY

(76) Inventor: Alfred P. Haus, Bushkill, PA (US)

Correspondence Address: GARCIA-ZAMOR INTELLECTUAL PROPERTY LAW;ATTN: RUY GARCIA-ZAMOR, 12960 LINDEN CHURCH ROAD, CLARKSVILLE, MD, 21029, US

(21) Appl. No.: 11/425,582

(22) Filed: Jun. 21, 2006

(51) Int. Cl. A22C 17/10; B65D 85/00; G01N 33/00

(52) U.S. Cl. 426/87; 426/129; 426/232

(57) ABSTRACT

Method and apparatus for tracking processed meat provided to the public from the time the raw meat was handled by an initial production facility.

### BACKGROUND

The present invention is generally directed to meat processing and, more specifically, to a method of tracking processed meat from the time of handling at an initial production facility.

Processed meat that is sold to the public on a large scale is handled by two separate production facilities. An initial production facility handles batches of raw meat and related ingredients that typically weigh in excess of ten thousand pounds. The meat is typically processed and separated into smaller portions that are shipped to a secondary production facility.

Current processed meat tracking systems only track meat from the secondary production facility forward. This can make it more difficult to trace a saleable portion back to an original batch of meat. Current meat tracking systems also use inefficient methods of tracking meat which includes recording, possibly by hand, each portion of meat used by the secondary production facility.

It would be advantageous to provide a method an apparatus for tracking meat that starts with the handling of the meat at the initial production facility; that uses an improved method of applying unique package identifiers; or that provides increased efficiency in the processing of data corresponding to meat shipped by the initial production facility.

### SUMMARY

Briefly speaking, one embodiment of the present invention is directed to a method of tracking processed meat provided to the public from the time the raw meat was handled by an initial production facility. The method includes: providing a film configured to wrap food products; printing unique, sequential identifiers on the film to form a film with a plurality of labels thereon; providing a batch of meat to be processed; preparing a plurality of portions of processed meat from the batch of meat; wrapping each of the portions of processed meat with a portion of the film such that each portion has a separate label comprising one unique, sequential identifier to form a shippable meat product; recording the unique sequential identifiers of at least the first and last shippable meat product formed from the batch of meat to associate each shippable meat product from the batch with a separate unique identifier; transferring the shippable meat product to a secondary production facility for further processing; slicing the shippable meat product into multiple saleable meat portions; and storing data correlating the multiple saleable meat portions to the unique, sequential identifier of the corresponding shippable meat product so that saleable meat portions can be traced back to the batch of meat used at the initial production facility.

In another aspect, the present invention is directed to a method of tracking processed meat provided to the public from the time the raw meat was handled by an initial production facility. The method includes providing a film configured to wrap food products; printing unique, sequential bar codes on the film using an inkjet printer to form a film with a plurality of labels thereon; providing a batch of meat to be processed; preparing a plurality of portions of processed meat from the batch of meat; wrapping each of the portions of processed meat with a portion of the film such that each portion has a separate label comprising one unique, sequential identifier to form a shippable meat product; scanning the unique sequential bar codes of at least the first and last shippable meat product formed from the batch of meat to associate at least the first and last shippable meat products from the batch with a separate unique, sequential bar code; storing data corresponding to at least the first and last shippable meat products in a searchable database; extrapolating and storing the data corresponding to the remaining shippable meat products which originated from the batch of meat; transferring the shippable meat product to a secondary production facility for further processing; slicing the shippable meat product into multiple saleable meat portions; and storing data correlating the multiple saleable meat portions to the unique, sequential bar code of the corresponding shippable meat product so that saleable meat portions can be traced back to the batch of meat used at the initial production facility.

In another aspect, the present invention is directed to a method of tracking processed meat provided to the public from the time the raw meat was handled by an initial production facility. The method includes: printing unique, sequential bar codes on a film to form a film with a plurality of labels thereon; providing a batch of meat to be processed; preparing a plurality of portions of processed meat from the batch of meat; wrapping each of the portions of processed meat with a portion of the film such that each portion has a separate label comprising one unique, sequential identifier to form a shippable meat product; scanning the unique sequential bar codes of at least the first and last shippable meat product formed from the batch of meat to associate at least the first and last shippable meat products from the batch with a separate unique, sequential bar code; storing data corresponding to at least the first and last shippable meat products in a searchable database; extrapolating and storing the data corresponding to the remaining shippable meat products which originated from the batch of meat; and transferring the shippable ship meat product to a secondary production facility for further

### BRIEF DESCRIPTION OF THE DRAWINGS

The foregoing summary, as well as the following detailed description of the preferred embodiments of the present invention will be better understood when read in conjunction with the appended drawings. For the purpose of illustrating the invention, there are shown in the drawings embodiments which are presently preferred. It is understood, however, that the invention is not limited to the precise arrangements and instrumentalities shown. In the drawings:

FIG. 1A is a perspective view of a first preferred apparatus for tracking meat in an initial production facility according to the present invention;

FIG. 1B is a perspective view of a preferred apparatus used in a secondary production facility that enables saleable meat products to be tracked to an initial batch of meat at the initial production facility;

FIG. 2 is a perspective view of a second preferred apparatus for tracking meat in an initial production facility according to the present invention;

FIG. 3 is a flowchart of a first preferred method according to the present invention of tracking processed meat provided to the public from the time the raw meat was handled by an initial production facility;

FIG. 4 is a flowchart of a second preferred method according to the present invention of tracking processed meat provided to the public from the time the raw meat was handled by an initial production facility; and

FIG. 5 is a flowchart of a third preferred method according to the present invention of tracking processed meat provided to the public from the time the raw meat was handled by an initial production facility.

### DETAILED DESCRIPTION OF THE PREFERRED EMBODIMENTS

Certain terminology is used in the following description for convenience only and is not limiting. The words “right,” “left,” “top,” and “bottom” designate directions in the drawings to which reference is made. The words “inwardly” and “outwardly” refer to directions toward and away from, respectively, the geometric center of the apparatus for tracking meat and designated parts thereof. The term “initial production facility”, as used in the claims and corresponding portions of the specification, means “a production facility that initially handles raw meat in large quantities to produce processed meat to be supplied to secondary production facilities for further handling and/or processing prior to the processed meat being offered for sale to the public.” The term “meat”, as used in the claims and in the corresponding portions of the specification, means “any one or combination of pork, turkey, beef, fish, and any other suitable animal meat and associated parts thereof, such as cartilage, ligament, etc.” The term “searchable database”, as used in the claims and the corresponding portions of the specification, means “any one of a computer, a controller, a remote database, a processor, a circuit, an integrated circuit, a printed circuit board, or like devices capable of storing a data”. The term “portions of processed meat”, as used in the claims and in corresponding portions of the specification, means The language “at least one of ‘A’, ‘B’, and ‘C’,” as used in the claims and in corresponding portions of the specification, means “any group having at least one ‘A’; or any group having at least one ‘B’; or any group having at least one ‘C’;—and does require that a group have at least one of each of ‘A’, ‘B’, and ‘C’.” Additionally, the words “a” and “one” are defined as including one or more of the referenced item unless specifically stated otherwise. The terminology includes the words above specifically mentioned, derivatives thereof, and words of similar import.

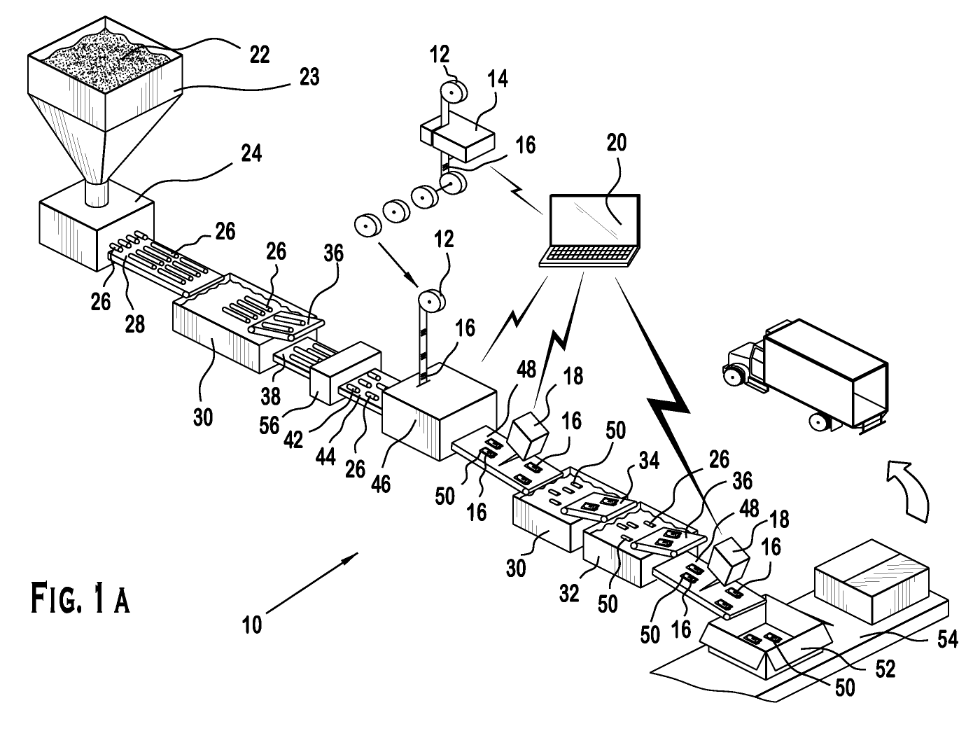

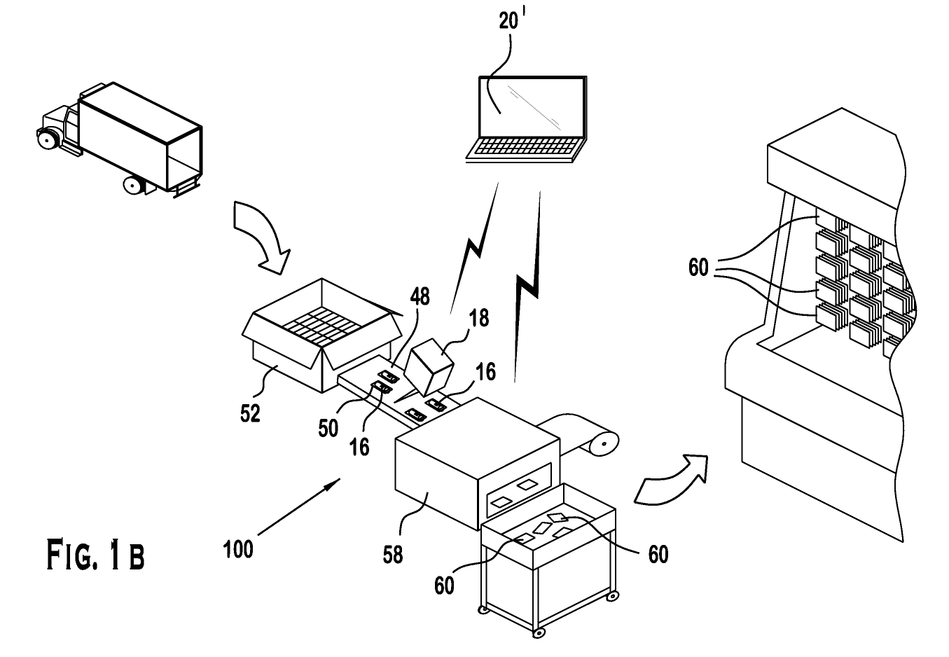

Referring to FIGS. 1-5, there are shown preferred embodiments of an apparatus 10 and methods for tracking meat. Briefly speaking, the present invention tracks meat from the point of initial handling by an initial production facility. By starting the tracking process from the initial handling of raw meat to be processed, the present invention simplifies the tracking of processed meat products sold to the public. It is also preferred, but not necessary, that data is only scanned for the first and last shippable meat products 50 (further described below) from a given batch of meat 22 while extrapolating the data that corresponds to the remaining shippable meat products from the same batch 22 which speeds up the manufacturing throughput and increases the efficiency of manufacturing due to minimizing the amount of scanning necessary to track all of the meat from a given batch of meat 22.

Referring to FIGS. 1A and 1B, an apparatus for tracking processed meat provided to the public from the time the raw meat 22 was handled by an initial production facility 10 is shown.

Referring to the middle, upper portion of FIG. 1A, a film 12 that is configured to wrap food products is provided. Unique (preferably sequential) identifiers 16 are printed on the film 12 to form a film 12 with a plurality of labels thereon. The identifiers 16 are preferably machine readable and may be a bar code or the like. The identifiers 16 are preferably positioned on the film 12 by a printer 14. The printer is preferably an inkjet printer capable of printing on film 12 that is moving at high speed. The ink is preferable a quick drying ink and the film is preferably a flat, laminate which may include nylon. The printing of identifiers 16 on the film 12 can occur separately from the processing of the meat or be integrated into the apparatus for processing meat without departing from the scope of the present invention. In the apparatus of FIG. 1, the labeling of the film 12 with identifiers 16 is separate from the processing of the meat 22.

A batch of meat 22 which may include additional seasonings or flavorings is held in a hopper 23. The meat 22 is preferably processed into a plurality of portions of processed meat 26. The term processed, as used in the claims and the corresponding portions of the specification, means “at least one of partially cooked, partially chilled, partially shaped, and packaged” The portions of processed meat 26 are preferably, but not necessarily, elongated logs of meat. The logs of meat are preferably moved by a conveyor which has a rack suspended therefrom for holding the logs.

The portions of processed meat 26 are then conveyed to the next processing station via a conveyor 28. Although conveyors 28, 34, 36, 38, 44, 48, and 54 are shown as endless belt conveyors, any suitable type of conveyor can be used without departing from the scope of the present invention. The elongated portions of processed meat 26 are then preferably at least partially cooked in a cooker 30. The portions of meat 26 are then preferably conveyed to a cutting station 56 that slices the preferred elongated logs of meat into smaller loaves of meat 26. The loaves of meat are preferably held in a tray with an array of cavities therein each shaped to hold a loaf of meat.

Each of the portions of processed meat (i.e., the preferred smaller loaves of meat) 26 is then wrapped by a packaging/sealing station. The portions of processed meat 26 are preferably wrapped with a portion of the film 12 such that each portion 26 has a separate label comprising one unique, sequential identifier 16 to form a shippable meat product 50.

The unique sequential identifiers 16 of at least the first and last shippable meat product 50 formed from the batch of meat 22 are recorded to associate each shippable meat product 50 from the batch 22 with a separate unique identifier 16. The identifiers 16 can be manually recorded and entered into a written log or a searchable database 20. Preferably the identifiers 16 are machine read by scanners 18.

The shippable meat products 50 are then preferably further cooked in a second cooker 30. The cookers 30 are preferably either hot water or steam cookers. However, any suitable cooking mechanism can be used without departing from the scope of the present invention. Then the shippable meat products 50 are preferably chilled in a chiller 32. The chiller is preferably a cold water chiller, but any suitable chilling/refrigeration/freezing mechanism can be used as part of the apparatus. The recording of the identifiers 16 on the shippable meat products 50 can occur anytime after wrapping or at multiple locations throughout the processing assembly.

The shippable meat product are preferably transferred to a secondary production facility 100 for further processing. The packaging 52 of the meat products 50 is removed to allow the shippable meat products to be identified by their labels/identifiers 16 and then further processed. The identifiers are preferably machine read by a scanner that is communication with a searchable database 20′. The databases of the initial and second production facilities can be integrated or in communication with each other without departing from the scope of the present invention.

The shippable meat products 50 are preferably sliced into multiple saleable meat portions 60 by a further processing station 58. The further processing station 60 can comprise one or more processing stages for slicing, sorting, packaging, mixing, seasoning, cooking, cooling, labeling, scanning, and/or sterilizing saleable meat portions. Data is stored correlating the multiple saleable meat portions 60 to the unique, sequential identifier 16 of the corresponding shippable meat product 50 so that saleable meat portions 60 can be traced back to the batch of meat 22 used at the initial production facility.

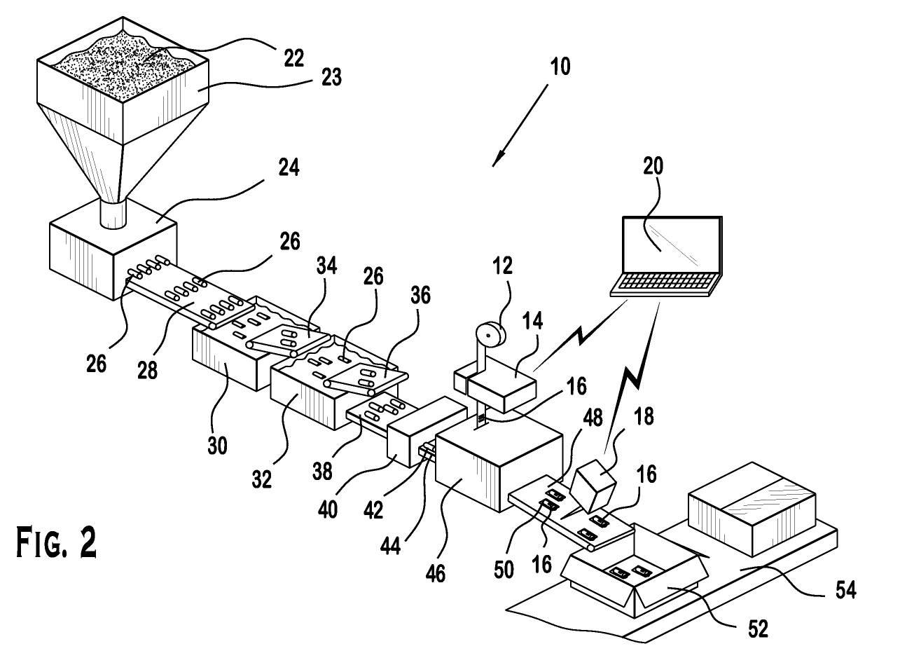

Referring to FIG. 2, an alternative preferred embodiment of an apparatus 10 for tracking meat in an initial production facility is shown. The description of particular components or methods of operations of any of the apparatuses shown in FIGS. 1A, 1B, and 2 can be generally applied to similar components in the other disclosed apparatus without departing from the scope of the present invention unless stated otherwise. That is the description of the film 12 used in the apparatus of FIG. 2 can generally apply to the film 12 of FIG. 1.

Referring specifically to FIG. 2, meat 22 that is preferably raw is initially positioned in a hopper 23 or similar storage container. The meat can be entirely from one type of animal or can be a blended product of different types of animal meat. Depending on the desired properties of the processed meat, the batch of meat 22 may include animal fat, ground animal bone, animal cartilage, etc. The batch of meat 22 may also include additional processed food components, or ingredients, such as spices, water, salt, pepper, flavorings, additives, flavor enhancers, and preservatives included in the batch of meat.

Meat 22 can be fed from the hopper 23 to a mold extruder 24 or the like. The mold 24 produces portions of processed meat 26 for further handling by the apparatus 10.

A conveyor 28 preferably transports the portions of processed meat 26 to a cooking station 30. Although the conveyors 28, 34, 36, 38, 44, 48, 54 shown are endless belt conveyors, those of ordinary skill in the art will appreciate from this disclosure that any suitable type of conveyor can be used without departing from the scope of the present invention. For example, one or more of the conveyors can be any one of an chain conveyor, a roller conveyor, a plat conveyor, a vibratory conveyor or the like.

The cooking station 30 is preferably a hot water cooker and/or a steam oven. However, those of ordinary skill in the art will appreciate from this disclosure that any suitable cooking apparatus can be used without departing from the scope of the present invention. After the portions of processed meat 26 have been at least partially cooked, the portions 26 are transported by a conveyor 34 to a chilling and/or cooling station 32. The cooling station 32 can use water, refrigeration, or driven air to cool the portions of processed meat 26.

A conveyor 38 may be used to transport the portions of meat 26 through an optional casing stripping station 32. The casing stripping station 32 can be used to remove a hardened outer layer from the portion of meat 26 prior to packaging.

The portions of meat 26 are then preferably conveyed to a packaging and/or sealing station 46 via conveyor 42. The packaging station 46 may use a hot fill process to generate shippable meat product 50 or may use irradiation or any other suitable method known for packaging meat in compliance with local food regulations.

Film 12 is preferably provided to a printer, such as an inkjet printer or the like, that is configured to place multiple unique identifiers 16 on the film. The film preferably, buy not necessary is fed through the printer 14 and to the packaging and/or sealing station at a high rate of speed. The film is preferably a laminate, flat film 12 and is preferably formed in part by nylon. However, those of ordinary skill in the art will appreciate from this disclosure that the film 12, can be formed of any suitable material without departing from the scope of the present invention. The unique identifiers 16 are preferably sequentially numbered and may be machine readable. It is preferred that the unique identifiers 16 are in the form of bar codes. It is preferred, but not necessary, that the ink is a quick drying ink. While FIG. 2 shows the film 12 being processed and labeled during the processing of the meat 26, it is more preferred that the film 12 is processed to have unique identifiers 16 placed thereon in a process that is separate from the meat preparation process.

From the packaging and/or sealing station 46 comes wrapped portions of processed meat each preferably bearing a unique identifier 16 that are shippable meat products 50. Alternatively, the meat portions 50 coming from the packagain and/or sealing station 46 can be transported through a separate sterilization station.

The unique identifiers 16 on the shippable meat products 50 are preferably recorded and stored on a searchable database 20 that associates the shippable meat product and its unique identifier 16 with the batch of meat 22 from which the shippable meat product 50 originated. The data can be manually noted and then entered into a manual log or a searchable database 20. It is preferred that the data corresponding to the shippable meat products 50 is read by a machine such as a bar code or CCD scanner or the like. Then, the data is preferably stored in a searchable database 20.

The apparatus 10 preferably scans at least the first and last shippable meat products 50 generated from a given batch of meat 22. This allows the database, or processor, 20 to extrapolate the data for the remaining sequentially numbered shippable meat products 50. The shippable meat products are then preferably packaged for shipping to a secondary production facility in boxes 52 or via any suitable transportation method.

Preferred methods of the present invention are described below. Unless stated otherwise, the apparatus used with the methods is that described above or an apparatus similar to that described above. The above described apparatus is exemplary. Those of ordinary skill in the art will understand that any suitable apparatus 10 can be used to perform the methods of the present invention. The steps of the methods of the present invention can be performed in any order unless specifically stated otherwise. For example, the processing of the film 12 to include identifiers 16 can be done prior to, during, or after the wrapping of the meat 26 in the film 12. Additional, steps in the preferred methods of the invention can be omitted without departing from the scope of the present invention.

The first preferred method of tracking processed meat provided to the public from the time raw meat was handled by an initial production facility follows. Unless otherwise stated similar steps in other preferred methods of the present invention are performed in generally the same manner.

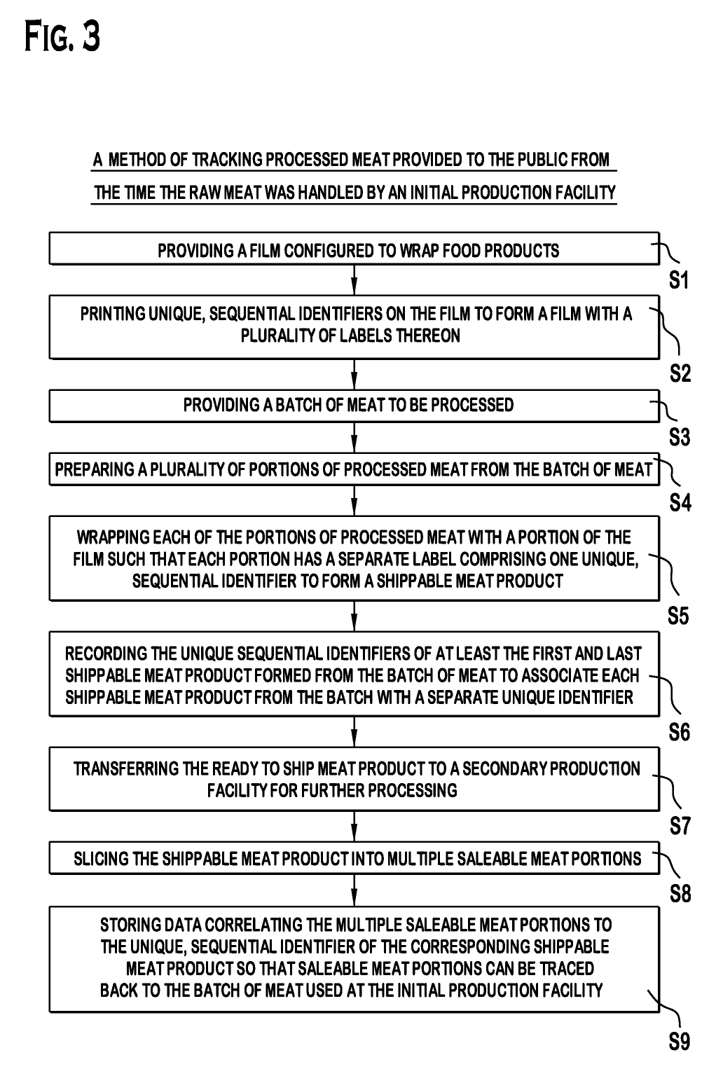

Referring to FIG. 3, the first preferred method includes providing a film 12 configured to wrap food products is provided during step S1. The film 12 is preferably handled and processed at a high speed. In step S2, unique, sequential identifiers 16 are printed on the film 12 to form a film with a plurality of labels 16 thereon. The printing is preferably performed with an inkjet printer which prints unique, sequential identifiers 16 on the film. The identifiers 16 are preferably bar codes or similar machine readable code.

A batch of meat 22 to be processed is provided during step S3. In step S4, a plurality of portions of processed meat 26 are prepared from the batch of meat 22.

Preparing the portions of processed meat 26 may include placing the portions 26 in an rack that encloses the portions of processed meat 26. Then, portions 26 of the batch of meat 22 may be heated in at least one of hot water and a steam oven 30. Afterwards, the portions of processed meat 26 can be cooled.

During step S5, each of the portions of processed meat 26 is preferably wrapped with a portion of the film 16 such that each portion 26 has a separate label 16 comprising one unique, sequential identifier 16 to form a shippable meat product 50. In step S6, the unique sequential identifiers 16 of at least the first and last shippable meat products 50 formed from the batch of meat 22 are recorded to associate each shippable meat product 50 from the batch 22 with a separate unique identifier 16. The identifiers 16 are preferably bar codes or the like. This step may include storing data corresponding to at least the first and last shippable meat products 50.

Step S6 may also include extrapolating and storing the data corresponding to the remaining shippable meat products 50 which originated from the batch of meat 22. For example if four hundred shippable meat products 50 are labeled with sequential unique identifiers 16 then, by knowing the identifiers 16 of the first and last shippable meat products 50, the range of shippable meat products 50 generated from a single batch of meat 22 can be determined. That is if the first shippable meat product 50 is labeled 1045 and the last shippable meat product is labeled 1445. then any shippable meat products labeled in the range of 1045 through 1445 originated from the same batch of meat 22. By reducing the number of shippable meat products 50 that need to be scanned to fully track all of the shippable meat products 50 from a batch of meat 22, manufacturing throughput and efficiency can be dramatically increased.

During step S7, the ready to ship meat products 50 are transferred to a secondary production facility for further processing. The secondary production facility may be a seller of sliced or smaller portioned meat, such as maker of frozen dinners or pre-sliced cold cuts or the like. The secondary production facility may process the shippable meat products 50 into smaller, saleable portions which may include further packaging or processing.

During step S8, shippable meat product 50 is sliced and/or processed into multiple saleable meat portions. The processing may include packaging the saleable meat portions. In step S9, data correlating the multiple saleable meat portions to the unique, sequential identifier 16 of the corresponding shippable meat product 50 is stored, preferably in a searchable database, so that saleable meat portions can be traced back to the batch of meat 22 used at the initial production facility.

The method may include identifying the batch of meat 22 that corresponds to a particular saleable meat portion by comparing the unique, sequential identifier 16 of the particular shippable meat product 50 used to manufacture the particular saleable meat product. This greatly facilitates tracking meat and simplifies the recall of contaminated saleable meat products. When studying the supply chain for processed meat for whatever reason, stored data can be reviewed to track the handling of the meat 22 from initial handling until sale to a customer.

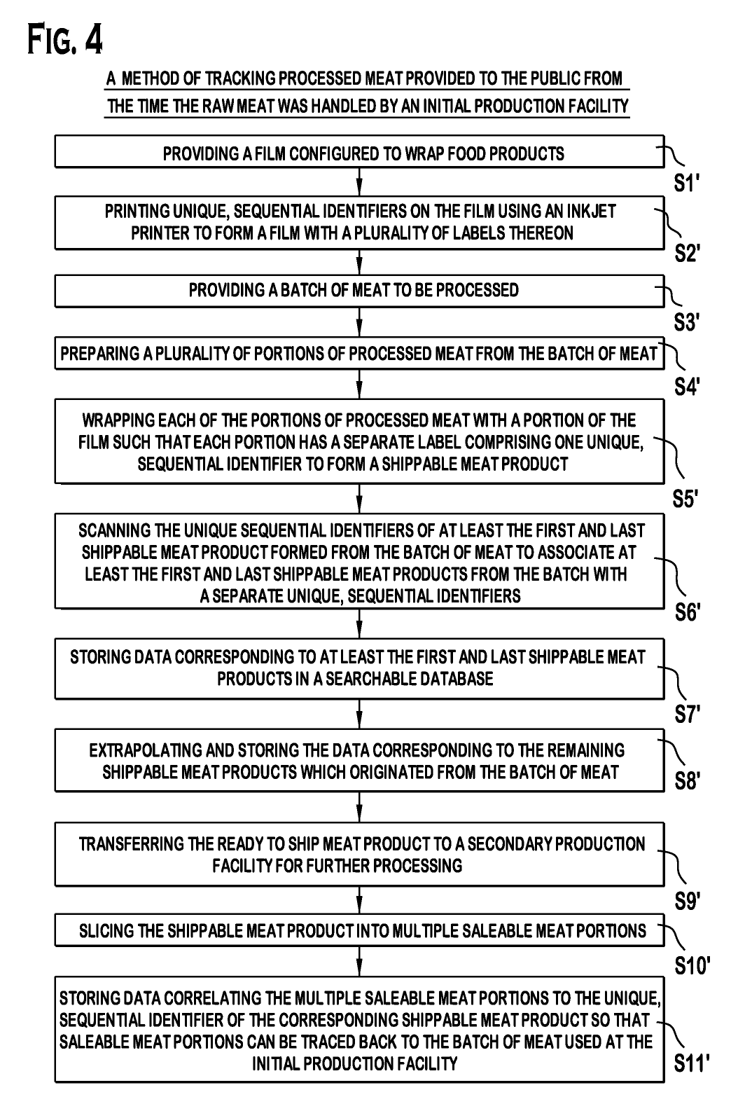

Referring to FIG. 4, a second preferred method of tracking processed meat provided to the public from the time the raw meat was handled by an initial production facility is shown. During step S1′, A film 12 is provided that is configured to wrap food products. In step S2′, unique, sequential bar codes 16 are printed on the film 12 using an ink jet printer 14 while the film 12 is moving at a high speed to form a film 12 with a plurality of labels 16 thereon.

In step S3′, a batch of meat 22 to be processed is provided. A plurality of portions of processed meat 26 are processed from the batch of meat 22 in step S4′.

Each of the portions of processed meat 26 is preferably wrapped with a portion of the film 12 such that each portion 26 has a separate unique label comprising one unique, sequential identifier to form a shippable meat product 50 during step S5′. In step S6′, The unique sequential bar codes 16 of at least the first and last shippable meat products 50 from the batch 22 are scanned to associate at least the first and last shippable meat products 50 from the batch 22 with a separate, unique, sequential bar code 16.

Data corresponding to the at least the first and last shippable meat products 50 are stored in a searchable database 20, during step S7′. In step S8′, data corresponding to the remaining shippable meat products 50 is extrapolated and stored. The shippable meat products 50 are then shipped to a secondary production facility for further processing during step S9′. The shippable meat product 50 is then divided into multiple saleable meat portions.

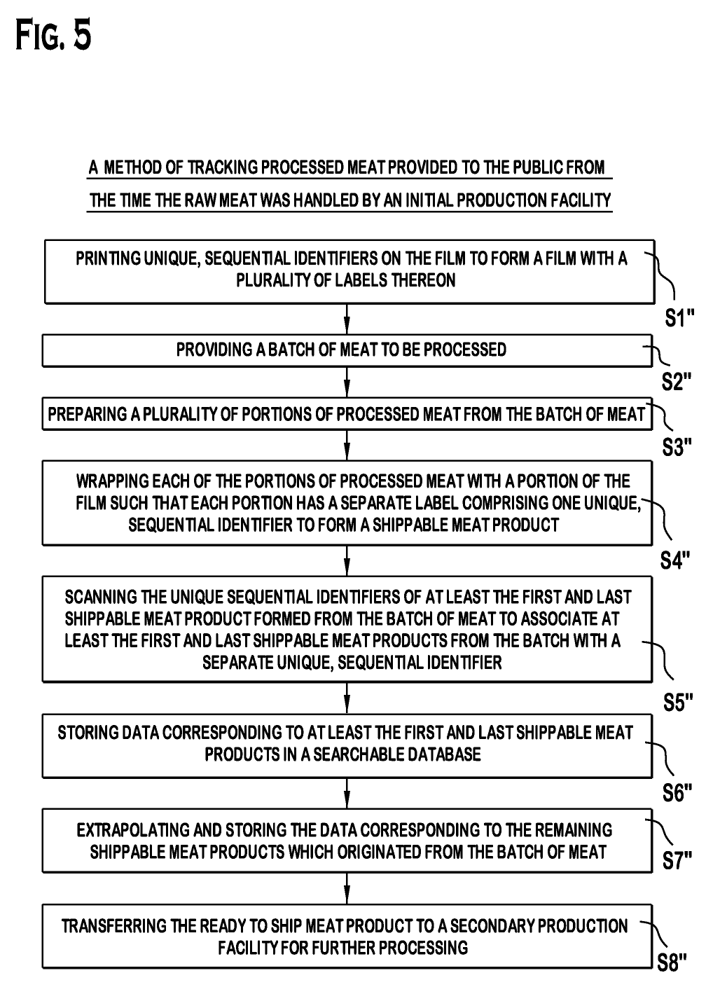

Referring to FIG. 5, a third preferred method of tracking processed meat provided to the public from the time the raw meat was handled by an initial production facility is shown. Unique, sequential bar codes 16 are printed on a film 12 to form a film 12 with a plurality of labels thereon, during step S1″. During step S2″, a batch of meat 22 to be processed is provided. In step S3″, a plurality of portions of processed meat 26 are prepared from the batch of meat 22. In step S4″, each of the portions of processed meat 26 are preferably wrapped with a portion of the film so that each portion 26 has a separate label including one unique, sequential identifier 16 to form a shippable meat product 50.

During step S5″, the unique, sequential bar codes 16 of at least the first and last shippable meat products 50 formed from the batch of meat 22 are scanned to association at least the first and last shippable meat products 50 from the batch 22 with a separate unique bar code 16. In step S6″, Data corresponding to at least the first and last shippable meat products 50 are stored in a searchable database. During step S7″, data corresponding the remaining shippable meat products 50 which originated from the same batch of meat 22 are calculated by extrapolation and stored. The calculation and storage is preferable automatically performed by the database 20. In step S8″, the shippable meat product 50 transferred to a secondary production facility for further processing.

The steps of any one of the above three preferred methods can be interchanged without departing from the scope of the present invention. It is recognized by those skilled in the art that changes may be made to the above described methods and/or apparatus without departing from the broad inventive concept thereof. It is understood, therefore, that this invention is not limited to the particular embodiments disclosed, but is intended cover all modifications which are within the spirit and scope of the invention as defined by the above specification, the appended claims and/or shown in the attached drawings.

What is claimed is:

1. A method of tracking processed meat provided to the public from the time the raw meat was handled by an initial production facility, comprising:
providing a film configured to wrap food products;
printing unique, sequential identifiers on the film to form a film with a plurality of labels thereon;
providing a batch of meat to be processed;
preparing a plurality of portions of processed meat from the batch of meat;
wrapping each of the portions of processed meat with a portion of the film such that each portion has a separate label comprising one unique, sequential identifier to form a shippable meat product;
recording the unique sequential identifiers of at least the first and last shippable meat product formed from the batch of meat to associate each shippable meat product from the batch with a separate unique identifier; and
transferring the shippable meat product to a secondary production facility for further processing;
slicing the shippable meat product into multiple saleable meat portions; and
storing data correlating the multiple saleable meat portions to the unique, sequential identifier of the corresponding shippable meat product so that saleable meat portions can be traced back to the batch of meat used at the initial production facility.

2. The method of claim 1, wherein the step of slicing the shippable meat further comprises packaging the saleable meat portions.

3. The method of claim 1, wherein the step of preparing a plurality of portions of processed meat further comprises:
placing portions of the batch of meat in an enclosed rack;
heating the portions of the batch of meat in at least one of hot water and a steam oven; and
cooling the portions of the batch of meat.

4. The method of claim 1, further comprising;
storing data corresponding to at least the first and last shippable meat products;
extrapolating and storing the data corresponding to the remaining shippable meat products which originated from the batch of meat.

5. The method of claim 2, further comprising identifying the batch of meat that corresponds to a particular saleable meat portion by comparing the unique, sequential identifier of the particular shippable meat product used to manufacture the particular saleable meat product.

6. The method of claim 1, wherein the step of providing a film comprises providing a laminate, flat film comprising nylon.

7. The method of claim 6, wherein the method of printing comprises using an inkjet printer to print the unique, sequential identifiers on the film.

8. The method of claim 7, wherein the step of printing comprises printing machine readable indicia on the film to form unique, sequential identifiers.

9. The method of claim 7, wherein the step of printing comprises printing bar codes on the film.

10. The method of claim 4, further comprising reviewing stored data to track the handling of the meat from initial handling until sale to a customer.

11. The method of claim 4, further comprising storing information regarding components in the batch of meat, wherein the components comprise at least one of types of meat, spices, water, additives, and preservatives included in the batch of meat.

12. A method of tracking processed meat provided to the public from the time the raw meat was handled by an initial production facility, comprising:
providing a film configured to wrap food products;
printing unique, sequential bar codes on the film using an inkjet printer to form a film with a plurality of labels thereon;
providing a batch of meat to be processed;
preparing a plurality of portions of processed meat from the batch of meat;
wrapping each of the portions of processed meat with a portion of the film such that each portion has a separate label comprising one unique, sequential identifier to form a shippable meat product;
scanning the unique sequential bar codes of at least the first and last shippable meat product formed from the batch of meat to associate at least the first and last shippable meat products from the batch with a separate unique, sequential bar code;
storing data corresponding to at least the first and last shippable meat products in a searchable database;
extrapolating and storing the data corresponding to the remaining shippable meat products which originated from the batch of meat transferring the shippable meat product to a secondary production facility for further processing;
slicing the shippable meat product into multiple saleable meat portions; and
storing data correlating the multiple saleable meat portions to the unique, sequential bar code of the corresponding shippable meat product so that saleable meat portions can be traced back to the batch of meat used at the initial production facility.

13. The method of claim 12, further comprising identifying the batch of meat that corresponds to a particular portion of processed meat by comparing the unique, sequential bar code of the particular portion of processed meat with stored data.

14. The method of claim 12, further comprising shipping the saleable meat portions and using the unique, sequential identifier to track the handling of the saleable meat portion from initial wrapping until sale to a customer.

15. The method of claim 12, further comprising storing information regarding components of the batch of meat, wherein the components comprise at least one of type of meat, spices, water, additives, and preservatives.

16. A method of tracking processed meat provided to the public from the time the raw meat was handled by an initial production facility, comprising:
printing unique, sequential bar codes on a film to form a film with a plurality of labels thereon;
providing a batch of meat to be processed;
preparing a plurality of portions of processed meat from the batch of meat;
wrapping each of the portions of processed meat with a portion of the film such that each portion has a separate label comprising one unique, sequential identifier to form a shippable meat product;
scanning the unique sequential bar codes of at least the first and last shippable meat product formed from the batch of meat to associate at least the first and last shippable meat products from the batch with a separate unique, sequential bar code;
storing data corresponding to at least the first and last shippable meat products in a searchable database;
extrapolating and storing the data corresponding to the remaining shippable meat products which originated from the batch of meat; and
transferring the shippable meat product to a secondary production facility for further processing.

17. The method of claim 16, further comprising:
slicing the shippable meat product into multiple saleable meat portions; and
storing data correlating the multiple saleable meat portions to the unique, sequential bar code of the corresponding shippable meat product so that saleable meat portions can be traced back to the batch of meat used at the initial production facility.

18. The method of claim 16, further comprising identifying the batch of meat that corresponds to a particular portion of processed meat by comparing the unique, sequential bar code of the particular portion of processed meat with stored data.

19. The method of claim 17, further comprising shipping the saleable meat portions and using the unique, sequential identifier to track the handling of the saleable meat portion from initial wrapping until sale to a customer.

20. The method of claim 16, further comprising storing information regarding components of the batch of meat, wherein the components comprise at least one of type of meat, spices, water, additives, and preservatives.
